(19) United States
(12) Patent Application Publication
(10) Pub. No.: US 2022/0305152 A1
(43) Pub. Date: Sep. 29, 2022

(54) Device for Sanitizing Air Filtering Headwear

(71) Applicant: Hall Labs LLC, Provo, UT (US)

(72) Inventors: Michael Hall, Provo, UT (US); David R. Hall, Provo, UT (US); Anthony E. Pullen, Tucson, AZ (US)

(73) Assignee: Hall Labs LLC, Provo, UT (US)

(21) Appl. No.: 17/209,216

(22) Filed: Mar. 23, 2021

(51) Int. Cl. A61L 2/10; A61L 2/04; A61L 2/22

(52) U.S. Cl. CPC A61L 2/10; A61L 2/04; A61L 2/22; A61L 2202/11; A61L 2202/14; A61L 2202/17; A61L 2202/15; A61L 2202/26; A62B 18/04

(57) ABSTRACT

A device to disinfect air filtering headwear comprising a chamber, an access port, a first disinfectant emitter to disinfect an outer surface of the headwear, and a mount wherein personal protection headwear is placed on, the mount comprises a second disinfectant emitter to disinfect an inner surface of the headwear.

### CROSS-REFERENCE TO RELATED APPLICATIONS

This application claims priority to U.S. Provisional Patent Application No. 62/992,277 titled “Head Covering Device” filed on Mar. 20, 2020, U.S. Provisional Patent Application No. 63/053,519 titled “Head Covering Device with Negative Air Flow”, U.S. Provisional Patent Application No. 63/053,523 titled “Head Covering Device with Environmental Control”, U.S. Provisional Patent Application No. 63/053,526 titled “Head Covering Device with a Communication Component”, U.S. Provisional Patent Application No. 63/053,537 titled “Head Covering Device with Automatic Air Moving System”, U.S. Provisional Patent Application No. 63/053,542 titled “Head Covering Device with Shroud”, U.S. Provisional Patent Application No. 63/053,546 titled “Head Covering Device with Washable Filtering Fabric”, U.S. Provisional Patent Application No. 63/053,548 titled “Head Covering Device with Electromagnetic Radiation Filtering Face Shield”, U.S. Provisional Patent Application No. 63/053,552 titled “Protective Mask with Negative Air Flow” filed on Jul. 17, 2020, and U.S. Provisional Patent Application No. 63/105,830 titled “Head Covering Device” filed on Oct. 26, 2020, which are incorporated herein by reference in their entirety.

### TECHNICAL FIELD

The present disclosure relates to sanitation devices.

### BACKGROUND

Personal protective equipment (PPE) on the market is typically designed for prevention of injury or infection. PPE is used by firefighters, policemen, HAZMAT specialists, health care workers, and other first responders. In many instances, the PPE is re-used or shared by the same or different person. The PPE may not be properly sanitized before being used in environments where the user encounters others with weakened immune systems, such as in nursing homes.

### SUMMARY

One aspect of the present invention is a device to disinfect air filtering headwear comprising a chamber, an access port, a first disinfectant emitter to disinfect an outer surface of the headwear, and a mount wherein personal protection headwear is placed on, the mount comprises a second disinfectant emitter to disinfect an inner surface of the headwear.

In another aspect of the invention, the headwear filters air that is inhaled by a user and filters air that is exhaled by the user.

In still another aspect, the outer disinfectant emitter emits UV light or a chemical disinfectant agent. The UV light can be UV-C light with a wavelength range of about 200-300 nm and the chemical disinfectant agent may be selected from the group consisting of a low level disinfectant, intermediate level disinfectant, high-level disinfectant, instrument grade, or hospital grade disinfectant to kill bacterial, fungal, and viral-based microorganisms, alcohols, aldehydes, oxidizing agents, peroxy and peroxo acids, phenolics, polymers, quarternary ammonium compounds, chlorine-based, iodine-based, acidic-based, basic-based, and metal-based inorganic compounds.

In a still further aspect, the device further comprises a heating element to add heat to the chamber.

In a yet still further aspect, the inner disinfectant emitter emits UV-C light with a wavelength range of about 200-300 nm.

In another aspect, the device further comprises a plurality of disinfectant emitters place uniformly throughout the chamber to uniformly disinfect the outer surface of the headwear.

In another aspect of the invention, the mount is in the shape of a human head or spherical-shape to expand and eliminate folds in the headwear for optimal disinfection of the inner surface of the headwear.

In still another aspect, the device further comprises a door or lid to seal the access port.

In a still further aspect, the device further comprises electronic controls to control the disinfectant being emitted from the outer disinfectant emitter and from the inner disinfectant emitter according to a pre-determined process. The electronic controls further control a heating element to provide heat to the chamber. The electronic controls control the amount and time of disinfectant and heat that is applied according to a pre-determined disinfection process to sufficiently eliminate specific microorganisms wherein the microorganisms are fungi, viruses, and bacteria. The electronic controls lock a door or lid to the access port when the pre-determined disinfection process is initiated and until it is completed, and when after the pre-determined disinfection process is completed a rinse solution is emitted into the chamber to remove residual or excess disinfectant. The process further comprises a fan to cool down or dry the chamber after the pre-determined disinfection process is completed.

In a still yet further aspect, the device further comprises a base with a plurality of apertures where the mount is attached to drain the disinfectant through the apertures.

In another still yet further aspect, the device further comprises one or more transparent windows with a UV light filter to view the personal protection equipment being sanitized in the chamber.

In another aspect of the invention, the device further comprises one or more additional mounts to disinfect a plurality of headwear simultaneously.

Further aspects and embodiments are provided in the following drawings, detailed description, and claims. Unless specified otherwise, the features as described herein are combinable and all such combinations are within the scope of this disclosure.

### BRIEF DESCRIPTION OF THE DRAWINGS

The following drawings are provided to illustrate certain embodiments described herein. The drawings are merely illustrative and are not intended to limit the scope of claimed inventions and are not intended to show every potential feature or embodiment of the claimed inventions. The drawings are not necessarily drawn to scale; in some instances, certain elements of the drawing may be enlarged with respect to other elements of the drawing for purposes of illustration.

FIG. 1 is a view of a PAPR mask being placed into the chamber of a sanitation device, according to an embodiment of the disclosure.

FIG. 2 is a view of a sanitation device in operation sanitizing a PAPR mask, according to an embodiment of the disclosure.

FIG. 3 illustrates various types of air filtering personal protection headwear that can be sanitized, according to an embodiment of the disclosure.

### DETAILED DESCRIPTION

### Overview

Embodiments of methods, materials and processes described herein are directed towards sanitizing devices. Sanitizing devices can be used to sanitize personal protection headwear to prevent the user from being infected with a contagious disease from a microorganism by a prior user or when the headwear is contaminated from use in an environment with infected people.

Sanitizing devices include components that emit one or more disinfectants. One type of disinfectant that may be emitted is UV-C light. Another type of disinfectant is a chemical-based disinfectant. Heat may also be applied to the headwear from the device. The disclosure herein describes designs and methods to sanitize rigid and flexible components of personal protection headwear. This includes a chamber with a holder that can emit disinfectants to sanitize an inner surface and outer surface of personal protection headwear.

### Definitions

The following description recites various aspects and embodiments of the inventions disclosed herein. No particular embodiment is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

The following terms and phrases have the meanings indicated below, unless otherwise provided herein. This disclosure may employ other terms and phrases not expressly defined herein. Such other terms and phrases shall have the meanings that they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly dictates otherwise. For example, reference to “a substituent” encompasses a single substituent as well as two or more substituents, and the like.

As used herein, “for example,” “for instance,” “such as,” or “including” are meant to introduce examples that further clarify more general subject matter. Unless otherwise expressly indicated, such examples are provided only as an aid for understanding embodiments illustrated in the present disclosure and are not meant to be limiting in any fashion. Nor do these phrases indicate any kind of preference for the disclosed embodiment.

As used herein, the term “user” refers to any individual who uses an HCD.

As used herein, the term “filter,” as a noun, refers to a device, typically composed of fibrous or porous materials which removes unwanted components, usually in the form of particulates, such as dust, pollen, mold, viruses, and bacteria, from air. Filters containing an adsorbent or catalyst, such as charcoal (carbon), may also remove odors and gaseous pollutants such as volatile organic compounds or ozone. Air filters are generally used in applications where air quality is important. As a verb, “filter” refers to the act of removing particles from air.

As used herein, the term “transparent” is used in its normal sense, that is the property of allowing light to pass through so that behind can be distinctly seen therethrough. The transparent components described and defined below are preferably clear, but may be tinted, in whole or in part.

The term “negative air flow” is used to indicate that, in accordance with embodiments of the invention, air is actively pulled inside the HCD through a filtering fabric by an air mover and the air exhausted out a filter in the air mover.

The term “positive air flow” is used to indicate that, in accordance with embodiments of the invention, air is actively pulled inside the HCD by an air mover through an inlet port in the air mover and exhausted through a filtering fabric.

The term “neutral air flow” is used to indicate that, in accordance with embodiments of the invention, a substantially static flow of air is maintained in the HCD. The air flow into and out of the HCD is controlled by the breathing in and breathing out of the user.

The term “head covering device” (HCD) refers to a device to cover the head of a user to protect the user from injury or infection. There are many forms of HCDs such as PAPRs, respirators, and helmets.

The term “personal protection equipment (PPE)” refers to protective clothing, helmets, goggles, air filtering headwear, or other garments or equipment designed to protect the wearer's body from injury or infection. The hazards addressed by protective equipment include physical, electrical, heat, chemicals, biohazards, and airborne particulate matter. Protective equipment may be worn for job-related occupational safety and health purposes, as well as for sports and other recreational activities. Protective clothing is applied to traditional categories of clothing, and protective gear applies to items such as pads, guards, shields, or masks, and others. PPE suits can be similar in appearance to a cleanroom suit. The purpose of PPE is to reduce employee exposure to hazards when engineering controls and administrative controls are not feasible or effective to reduce these risks to acceptable levels. PPE is needed when there are hazards present. PPE has the serious limitation that it does not eliminate the hazard at the source and may result in employees being exposed to the hazard if the equipment fails.

The term “PAPR” refers to a powered air-purifying respirator that is a type of respirator used to safeguard workers against contaminated air. PAPRs consist of a headgear-and-fan assembly that takes ambient air contaminated with one or more type of pollutant or pathogen, actively removes (filters) a sufficient proportion of these hazards, and then delivers the clean air to the user's face or mouth and nose. PAPRs have a higher assigned protection factor than filtering facepiece respirators such as N95 masks. PAPRs are sometimes called positive-pressure masks, blower units, or just blowers.

### Exemplary Embodiments

The present disclosure relates to devices to disinfect and sanitize PPE such as air filtering headwear. The headwear can be shared among health-care workers and first responders. As a result, there are several vectors where workers may infect each other or those they encounter, such as nursing home residents and hospitalized patients with weakened immune systems. The present disclosure illustrates embodiments of sanitation devices designed to sanitize various forms of air filtering headwear.

In various exemplary embodiments, the sanitizing device includes a chamber to place the headwear into. The chamber may be closed and provides emitters to emit disinfectants uniformly over the inner and outer surface of the headwear. The disinfectants include UV light or a chemical-based disinfectant.

In an exemplary embodiment, the device includes a spherically-shaped holder to place the air filtering headwear onto. The holder can emit UV light to sanitize the inner surface of the headwear. The sanitizing device can also emit heat into the chamber.

In various exemplary embodiments, the sanitizing device can be programmed to sanitize air filtering headwear in a pre-determined manner to ensure that the headwear is properly sanitized.

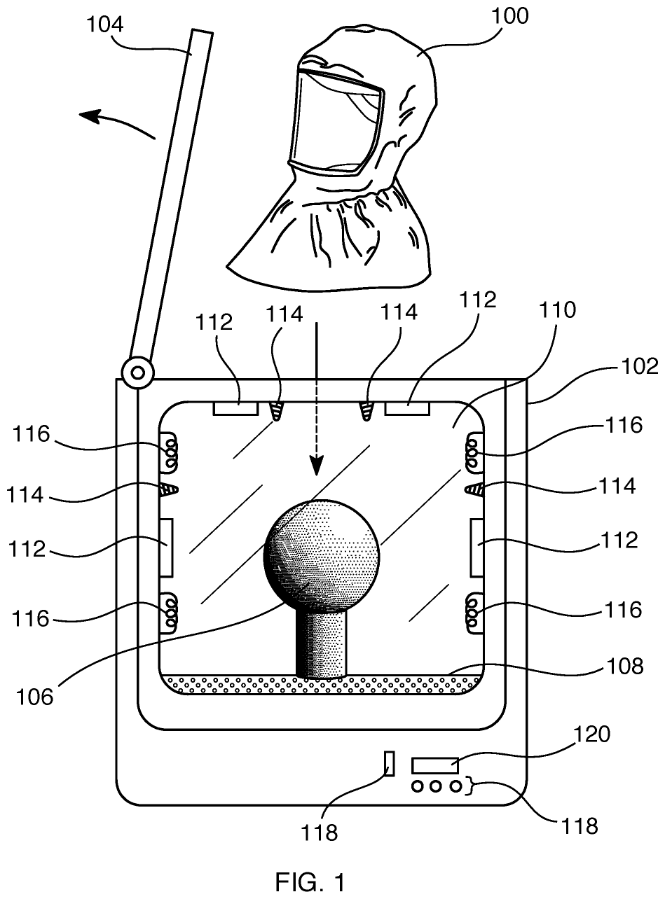

FIG. 1 is a view of a PAPR mask 100 being placed into the chamber of a sanitation device 102, according to an embodiment of the disclosure. Although a PAPR mask is illustrated in FIG. 1, it should be known that other type of air filtering, personal protection headwear may also be sanitized by the device described herein. In an exemplary embodiment, the headwear can filter intake and exhaust air.

The sanitation device is in the shape of a box, but other shapes are possible. The sanitation device includes a lid or door 104 that is opened to place the PAPR mask inside the device. The lid is connected to the device by a hinge or other flexible mechanism. The device includes a spherical-shaped holder 106 to place the PAPR mask on. In some embodiments, the holder may be in the shape of a human head.

The device further comprises a base 108 where the holder is mounted to. The base can be made of a metal, glass, or polymer, and further comprises a plurality of holes, apertures, or perforations to allow for drainage of a chemical-based disinfectant or rinse solution. The drained fluids may be collected to a fluid receptacle in the chamber below the base or outside of the chamber.

The device further comprises a transparent window 110 to allow a user to observe the sanitation process. In a preferred embodiment, the window includes a UV light filtering layer to prevent UV light from exiting the chamber. The layer may be a light polarizing film. In some embodiments, the device does not have a transparent window.

The sanitation device includes one or more UV light emitters 112. In a preferred embodiment, the UV light emitters emit UV-C light. UV-C light has a wavelength range of about 200-300 nm and is commonly used in disinfection devices to kill or inactive microorganisms in the food, air, and water industries. The light emitters are ideally uniformly in various locations throughout the chamber to ensure that a substantial amount of the PAPR mask surface is exposed to the UV-C light. Holder 106 is also capable of emitting UV-C light to irradiate the inner surfaces of the PAPR mask.

The sanitation chamber includes one or more disinfectant spray emitters 114. The spray emitters may emit one or more chemical agents such as a sterilant that is used to sterilize medical devices or instruments, a low level disinfectant, intermediate level disinfectant, high-level disinfectant, instrument grade, or hospital grade disinfectant to kill bacterial, fungal, and viral-based microorganisms. Such chemical agents include alcohols, aldehydes, oxidizing agents, peroxy and peroxo acids, phenolics, polymers, and quarternary ammonium compounds. The chemical agents may be chlorine-based, iodine-based, acidic-based, basic-based, or metal-based inorganic compounds. In other embodiments, the holder 106 may also comprise one or more disinfectant spray emitters.

The sanitation device further includes one or more resistive heating elements or coils 116 located in the chamber. The heating elements can provide heat to the chamber to further aid in sterilization of the PAPR mask.

It should be known that any of UV-C light emitters 112, disinfectant spray emitters 114, heating elements 116, or other sanitation methods are optional. Any combination of these three or other sanitation methods and devices may be used in the sanitation chamber. When two or more sanitation methods are used to clean the PAPR mask or other air filtering headwear, the methods may be carried out sequentially, in parallel, or a combination thereof. For example, a chemical agent may be sprayed on the PPE followed by irradiation with UV-C light, and then followed by the application of heat. The duration, temperature, amount of chemical agent and other factors may be carefully controlled in a pre-determined disinfection process depending on the type of headwear, the type of microorganism to be disinfected (e.g., fungi, viruses, bacteria), or if any sensitive or delicate devices are part of the headwear such as sensors, electric fans, or other types of environmental and communication control components.

The device further comprises controls 118 and a display 120 to set and control the duration of exposure of the PAPR mask to UV-C light, temperature level of the chamber, and whether to use a disinfectant spray. In some instances, a rinse may be emitted by an emitter in the chamber and applied to the surface to remove any residual or excess disinfecting chemical agent.

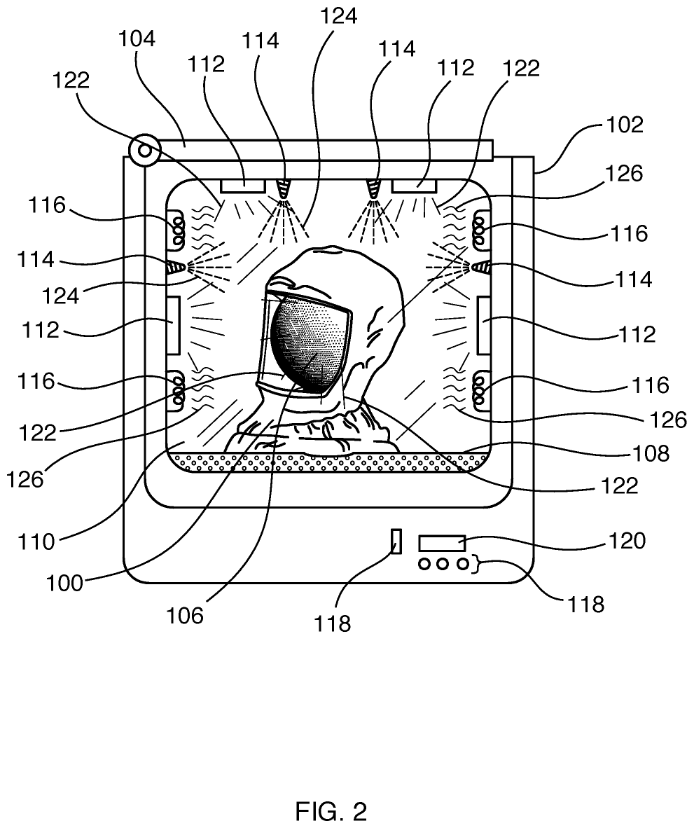

FIG. 2 is a view of a sanitation device 102 in operation sanitizing a PAPR mask 100, according to an embodiment of the disclosure. In this view, a PAPR mask has been placed in the chamber of the device and over the holder 106. The inside and top of the PAPR mask is resting on the top of the holder. The lid 104 is closed and the sanitation process has begun. Light emitters 112 are emitting UV-C light 122, disinfectant spray emitters 114 are emitting a disinfectant spray 124, and heating elements 116 are emitting heat 126 to heat the chamber and PAPR mask. The holder 106 is also emitting UV-C light 122 to sanitize the inside of the PAPR mask.

In a preferred embodiment, the sanitizing device provides a locking mechanism for the chamber lid such that when the sanitation process is initiated, the lid does not unlock until the pre-determined sanitation time and process is completed and the UV-C light is turned off. This prevents incomplete sanitation and exposure of the eyes of a user to damaging UV-C light or exposure to a chemical sanitizing agent if opening the device too early. The inside of the chamber may further comprise one or more fans to cool the chamber once the sanitation process is completed. Although only one PAPR mask is being placed into the chamber and being sanitized in FIGS. 1-2, in some embodiments the chamber may be large enough to comprise a plurality of holders to sanitize more than one PAPR mask or other type of air filtering headwear at a time.

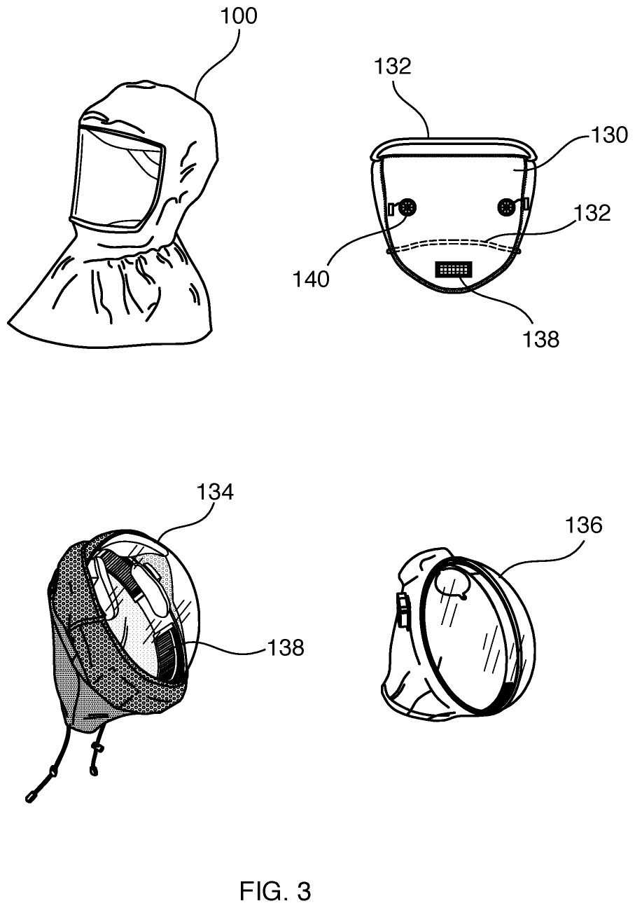

FIG. 3 illustrates various types of air filtering personal protection headwear that can be sanitized, according to an embodiment of the disclosure. As previously mentioned, various types of head covering PPE can be sanitized with the device described herein in addition to a PAPR mask 100. Face masks 130 that only cover the nose, the mouth, or eyes, or a combination thereof may be sterilized. The straps can be used to secure the mask to the holder 106. Headwear that filters both the intake and exhaust air 134, 136 may also be sanitized with a sanitation chamber described herein. Devices that provide filtered air to the user with negative, positive, or neutral air flow may be sanitized. Headwear that filters the inhaled air and filters the exhaled air can be sanitized. Headwear that has filters 138 such as N95, charcoal, paper, or pleated filters may be sanitized. Headwear with delicate electric fans 140, sensors, power supplies like batteries or solar cells, may also be sanitized by the sanitizing device described herein. Where there are filters or electronic devices, only UV-C light may be used to sanitize the headwear or where the heat supplied to the chamber by the heating elements is kept at a pre-determined minimum emission to sanitize but not damage the electronic devices.

It should be known that some sanitation devices have shelves to place items onto to be sanitized. The chamber disclosed herein provides a disinfectant holder that spreads apart and expands the fabric component of the air filtering headwear and allows for the inside surface to be sanitized.

The invention has been described with reference to various specific and preferred embodiments and techniques. Nevertheless, it is understood that many variations and modifications may be made while remaining within the spirit and scope of the invention.

What is claimed is:

1. A device to disinfect air filtering headwear comprising:
a chamber;
an access port;
a first disinfectant emitter to disinfect an outer surface of the headwear; and
a mount wherein personal protection headwear is placed on, the mount comprises a second disinfectant emitter to disinfect an inner surface of the headwear.

2. The device of claim 1, wherein the headwear filters air that is inhaled by a user and filters air that is exhaled by the user.

3. The device of claim 1, wherein the outer disinfectant emitter emits UV light or a chemical disinfectant agent.

4. The device of claim 3, wherein the UV light is UV-C light with a wavelength range of about 200-300 nm.

5. The device of claim 3, wherein the chemical disinfectant agent is selected from the group consisting of a low level disinfectant, intermediate level disinfectant, high-level disinfectant, instrument grade, or hospital grade disinfectant to kill bacterial, fungal, and viral-based microorganisms, alcohols, aldehydes, oxidizing agents, peroxy and peroxo acids, phenolics, polymers, quarternary ammonium compounds, chlorine-based, iodine-based, acidic-based, basic-based, and metal-based inorganic compounds.

6. The device of claim 1, further comprising a heating element to add heat to the chamber.

7. The device of claim 1, wherein the inner disinfectant emitter emits UV-C light with a wavelength range of about 200-300 nm.

8. The device of claim 1, further comprising a plurality of disinfectant emitters place uniformly throughout the chamber to uniformly disinfect the outer surface of the headwear.

9. The device of claim 1, wherein the mount is in the shape of a human head or spherical-shape to expand and eliminate folds in the headwear for optimal disinfection of the inner surface of the headwear.

10. The device of claim 1, further comprising a door or lid to seal the access port.

11. The device of claim 1, further comprising electronic controls to control the disinfectant being emitted from the outer disinfectant emitter and from the inner disinfectant emitter according to a pre-determined process.

12. The device of claim 11, wherein the electronic controls further control a heating element to provide heat to the chamber.

13. The device of claim 11, wherein the electronic controls control the amount and time of disinfectant and heat that is applied according to a pre-determined disinfection process to sufficiently eliminate specific microorganisms.

14. The device of claim 13, wherein the microorganisms are fungi, viruses, and bacteria.

15. The device of claim 13, wherein the electronic controls lock a door or lid to the access port when the pre-determined disinfection process is initiated and until it is completed.

16. The device of claim 13, wherein after the pre-determined disinfection process is completed a rinse solution is emitted into the chamber to remove residual or excess disinfectant.

17. The device of claim 16, further comprising a fan to cool down or dry the chamber after the pre-determined disinfection process is completed.

18. The device of claim 1, further comprising a base with a plurality of apertures where the mount is attached to drain the disinfectant through the apertures.

19. The device of claim 1, further comprising one or more transparent windows with a UV light filter to view the personal protection equipment being sanitized in the chamber.

20. The device of claim 1, further comprising one or more additional mounts to disinfect a plurality of headwear simultaneously.
